(19) United States
(12) Patent Application Publication
(10) Pub. No.: US 2025/0186733 A1
(43) Pub. Date: Jun. 12, 2025

(54) ABSORBER CANISTER AND MEDICAL DEVICE

(71) Applicant: GE Precision Healthcare LLC, Waukesha, WI (US)

(72) Inventors: Hui Fang, Wuxi (CN); Yiqiang Liu, Wuxi (CN)

(21) Appl. No.: 18/968,491

(22) Filed: Dec. 4, 2024

(30) Foreign Application Priority Data
Dec. 8, 2023 (CN) 202311690084.1

(51) Int. Cl. A61M 16/22

(52) U.S. Cl. CPC A61M 16/22

(57) ABSTRACT

Embodiments of the present application provide an absorber canister, the absorber canister comprising: a canister body including an accommodating chamber for accommodating an absorbent, wherein an opening is provided at an upper end of the canister body; a canister lid connected to the canister body, wherein the canister lid, in a closed state, closes the opening; a gas passage including the accommodating chamber, wherein a gas flow can flow through the absorbent in the accommodating chamber via the gas passage; and an elastic component disposed in the accommodating chamber, the elastic component being deformable by an external force to increase the volume of the accommodating chamber.

### CROSS-REFERENCE TO RELATED APPLICATIONS

This application is based on and claims priority to Chinese Application No. 202311690084.1, filed on Dec. 8, 2023, the entire contents of which is herein incorporated by reference.

### TECHNICAL FIELD

Embodiments of the present application relate to the technical field of medical devices, and relate in particular to an absorber canister and a medical device.

### BACKGROUND

An absorber canister is a container used to accommodate an absorbent in an anesthesia machine. In existing anesthesia machines, an absorber canister containing an absorbent is installed on a breathing circuit and used to process carbon dioxide (CO2) gas exhaled by a patient. Typically, the absorbent includes a mixture of sodium hydroxide (NaOH) particles and calcium oxide (CaO) particles.

The absorber canister is a closed chamber located in a gas passage of a breathing circuit system, and the absorbents react with the CO2 gas, effective alkaline substances are gradually depleted. Therefore, the absorbent in the absorber canister need to be replaced regularly. To facilitate the replacement of the absorbent, the absorber canister is usually provided with a canister lid.

It should be noted that the above introduction of the background is only set forth to help clearly and completely describe the technical solutions of the present application, and to facilitate the understanding of those skilled in the art.

### SUMMARY

The inventors have found that a scale line is provided on a canister body of an absorber canister to remind users not to exceed the scale line when filling the absorber canister with absorbent. However, during the actual operations of opening a canister lid and pouring in absorbent, users often fail to notice the scale line and unintentionally pour in too much absorbent. Consequently, the absorbent ends up forming a cone-shaped protrusion exceeding the scale line, preventing the canister lid from closing properly.

In view of at least one of the above technical problems, the embodiments of the present application provide an absorber canister, the absorber canister comprising:
A canister body comprising an accommodating chamber for accommodating an absorbent, wherein an opening is provided at an upper end of the canister body;
A canister lid connected to the canister body, wherein the canister lid, in a closed state, closes the opening;
A gas passage comprising the accommodating chamber, wherein a gas flow can flow through the absorbent in the accommodating chamber via the gas passage; and
An elastic component disposed in the accommodating chamber, the elastic component being deformable by an external force to increase the volume of the accommodating chamber.

The embodiments of the present application further provide a medical device having a breathing circuit, the absorber canister described above being disposed on the breathing circuit.

One of the beneficial effects of the embodiments of the present application is that: The elastic component is disposed in the absorber canister, so that the volume of the accommodating chamber for accommodating absorbent can be changed. Even if a user fills the absorber canister with too much absorbent, the canister lid can still be closed properly. In this way, it is convenient for the user to fill the absorber canister with absorbent, and the user experience is improved.

With reference to the following description and drawings, specific implementations of the embodiments of the present application are disclosed in detail, and the means by which the principles of the embodiments of the present application can be employed are illustrated. It should be understood that the implementations of the present application are not limited in scope thereby. Within the scope of the spirit and clauses of the appended claims, the implementations of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive effort. In the drawings:

FIG. 1 is a schematic diagram of an absorber canister according to an embodiment of the present application;

FIG. 2 is a cross-sectional view of the absorber canister according to an embodiment of the present application;

FIG. 3 is another cross-sectional view of the absorber canister according to an embodiment of the present application;

FIG. 4 is a schematic diagram of a bottom mesh of an absorber canister according to an embodiment of the present application;

FIG. 5 is another schematic diagram of the bottom mesh of an absorber canister according to an embodiment of the present application;

FIG. 6 is a schematic diagram of a canister lid of an absorber canister according to an embodiment of the present application; and

FIG. 7 is a schematic diagram of a medical device according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes one of associated listed terms or any and all combinations of a plurality of associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least partly on the basis of . . . ”, unless otherwise specified in the context.

The features described and/or illustrated for one implementation may be used in one or more other implementations in the same or similar manner, be combined with features in other implementations, or replace features in other implementations. The terms “include/comprise” when used herein refer to the presence of features, integrated components, steps, or assemblies, but do not preclude the presence or addition of one or more other features, integrated components, steps, or assemblies.

The following is a specific description of the embodiments of the present application with reference to the drawings.

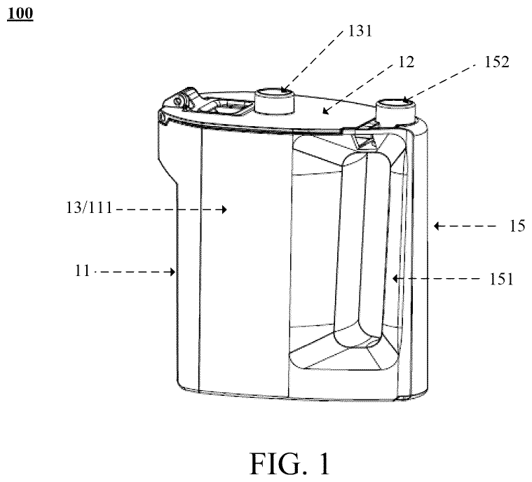

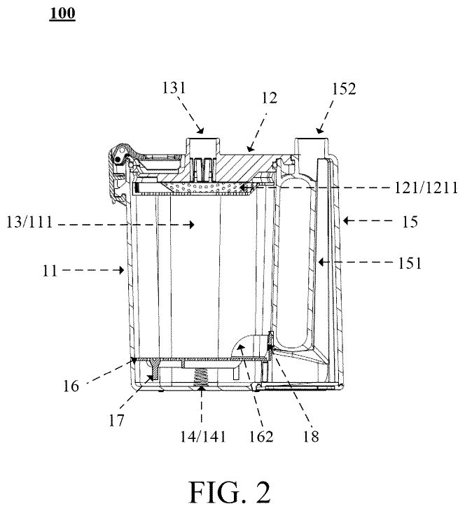

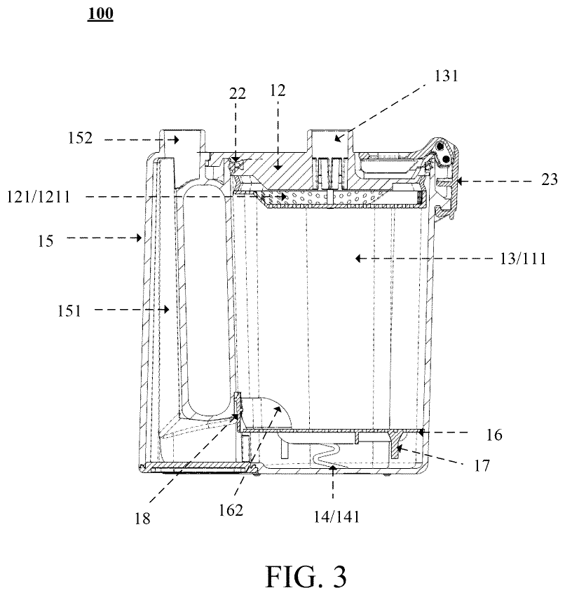

An absorber canister is provided in the embodiments of the present application. FIG. 1 is a schematic diagram of an absorber canister according to an embodiment of the present application. FIG. 2 is a cross-sectional view of the absorber canister. FIG. 3 is another cross-sectional view of the absorber canister. As shown in FIG. 1 to FIG. 3, the absorber canister 100 includes a canister body 11, a canister lid 12, a gas passage 13, and an elastic component 14 (not shown in FIG. 1).

The canister body 11 includes an accommodating chamber 111 for accommodating an absorbent, and an opening is provided at an upper end of the canister body 11.

The canister lid 12 is connected to the canister body 11, and the canister lid 12, in a closed state, closes the opening on the upper end of the canister body 11.

The gas passage 13 includes the accommodating chamber 111, and a gas flow can flow through the absorbent in the accommodating chamber 111 via the gas passage 13.

The elastic component 14 is disposed in the accommodating chamber 111, and the clastic component 14 is deformable by an external force F to increase the volume of the accommodating chamber 111.

According to the above embodiment, the elastic component is disposed in the absorber canister, so that the volume of the accommodating chamber for accommodating the absorbent can be changed. Even if a user fills the absorber canister with too much absorbent, the canister lid can still be closed properly. Specifically, when an excessive amount of absorbent is added, it is difficult to close the canister lid of a conventional absorber canister. This is because the height of the excessive amount of absorbent in the absorber canister is too high, and hinders closing of the canister lid. In the embodiment of the present application, the elastic component is deformable by an external force to increase the volume of the accommodating chamber. In this configuration, even if an excessive amount of absorbent is accommodated in the absorber canister, the canister lid, when being closed, can press the absorbent and eventually apply a pressing force to the clastic component, thereby eventually increasing the effective volume of the absorber canister for accommodating the absorbent. Therefore, it is possible to close the canister lid without taking out the excessive amount of absorbent, and the sealing performance of the absorber canister is not affected. In this way, it is convenient for the user to fill the absorber canister with absorbent, and the user experience is improved.

The accommodating chamber 111 is described below. The absorber canister 100 is a hollow cylindrical shape or another shape, and the accommodating chamber 111 is a chamber in the inner space of the absorber canister 100 and is used for accommodating absorbent. When the canister lid 12 is in the closed state, the accommodating chamber 111 may include a space enclosed by a sidewall of the canister body 11, the bottom of the canister body 11, and the canister lid 12. For example, the accommodating chamber 111 is a space enclosed by the sidewall of the canister body 11, a top mesh 121 (see the description below), and a bottom mesh 16 (see the description below). The elastic component 14 is disposed inside the accommodating chamber 111. For example, the elastic component 14 is disposed at the top or the bottom of the accommodating chamber 111, and is deformed by an external force F, so as to change the volume or the size of the accommodating chamber 111.

In some embodiments, the elastic component 14 is disposed at the bottom of the accommodating chamber 111, for example, at the bottom of the canister body 11. After an absorbent is added into the accommodating chamber 111, the absorbent can be accommodated in a space enclosed by the elastic component 14 and the sidewall of the canister body 11. It will be appreciated that, due to the weight of the absorbent itself, the elastic component 14 is allowed to undergo a certain deformation after the absorbent is added, but is configured to be able to balance the weight of the absorbent sufficiently without reaching the maximum deformation limit. In this case, even if an excessive amount of absorbent is added, during closing of the canister lid 12, the canister lid 12 can still transfer a downward pressing force to the elastic component 14 through the absorbent, to further deform the elastic component. The clastic component 14 disposed at the bottom of the accommodating chamber 111 can ensure the integrity of an outer surface of the absorber canister 100. In addition, under the protection of the sidewall of the canister body 11, a horizontal component of the downward pressing force on the canister lid 12 can be effectively offset by the sidewall of the canister body 11, so that the downward pressing force vertically acts on the elastic component 14 to the maximum extent, thereby ensuring the smoothness of deformation or recovery from deformation of the elastic component 14, and preventing the occurrence of jamming.

In some other embodiments, the elastic component 14 is disposed at the top of the accommodating chamber 111, for example, under the canister lid 12. In this way, during closing of the canister lid 12, if the height of the absorbent is too high, the absorbent can directly contact the elastic component 14 at the bottom of the canister lid 12. Pressing the canister lid 12 downward will further increase the magnitude of the supporting force of the absorbent for the elastic component 14 so as to cause deformation of the elastic component 14, thereby eventually increasing the volume of the accommodating chamber 111. The elastic component 14 disposed under the canister lid 12 has the advantage of being conducive to cleaning and disinfecting. When the canister lid 12 is open, the elastic component 14, which is originally under the canister lid 12, can be exposed upward. In this case, the user may easily clean and disinfect the elastic component 14. Especially in the case of an elastic component 14 with a complex structure, cleaning and disinfection thereof can be quite challenging, and the above-described design greatly reduces the difficulty of cleaning and disinfection.

In some embodiments, the elastic component 14 may further be configured in a combination of various manners. Two elastic components 14 disposed at the bottom and the top of the accommodating chamber 111 may be included. For example, two elastic components 14 disposed under the canister lid 12 and disposed at the bottom of the canister body 11 may be included. The specific configuration may be a configuration described in any embodiment in the present disclosure, and will not be repeated here. A combination of a plurality of elastic components can provide larger deformation room for the elastic component 14. Accordingly, more redundancy of the absorbent can be allowed.

It will be appreciated that the specific configuration of the elastic component 14 may be in a variety of manners in light of the teachings of the above embodiments of the present application. In some embodiments, any elastic component 14 that can deform under an external force as described above in the present application is allowed, and such an elastic component 14 can be used to increase the volume of the accommodating chamber so that excess absorbent can be accommodated without being removed. In addition, some other examples of the present application describe the specific configuration of the elastic component 14 in a preferred implementation, which is described in detail below.

The gas passage 13 is described below. The gas passage 13 includes the accommodating chamber 111. That is, the space in which the gas passage 13 is located includes at least the space in which the accommodating chamber 111 is located, or the space in which the gas passage 13 is located is greater than or equal to the space in which the accommodating chamber 111 is located. This configuration can ensure that gas in the gas passage 13 flows through the accommodating chamber 111 and can be in full contact with the absorbent in the accommodating chamber 111, and further, gas components to be absorbed (for example, carbon dioxide and water vapor) can sufficiently react with the absorbent so as to be absorbed.

In some embodiments, the canister body 11 is further provided with an indicator line L, the indicator line L being used to indicate the height of the absorbent. For example, the indicator line L is provided on an outer wall of the canister body 11, thereby facilitating the user's observation. For another example, one or more indicator lines L are provided. When there are a plurality of indicator lines L, the indicator lines L may be uniformly distributed on the outer wall of the canister body 11, and there is a maximum indicator line L_max for indicating the maximum height allowed for absorbent filling. The arrangement of the indicator line can more effectively inform the user whether the absorbent is in an excess state. In particular, the arrangement of the indicator line and the arrangement of the elastic component 14 can match each other. Specifically, although the elastic component 14 provides some fault tolerant space, and allows the user to add an excessive amount of absorbent, the space is still limited. If no indicator line is provided, it is difficult for the user to predict to what extent the addition of the absorbent will be considered excessive.

In an embodiment of the present application, the height of the indicator line Lis configured to match the deformation capacity of the elastic component 14. That is, the amount of absorbent corresponding to the height of the indicator line L may be configured such that the absorbent does not contact the canister lid 12 when in the closed state, and therefore, the canister lid 12 does not press the elastic component 14. In an actual usage scenario, the user may estimate an addition amount of absorbents by observing the indicator line L, and even if too much absorbent is added due to an operation error, the addition can be stopped in time through observation of the indicator line L. In that case, the amount of absorbent exceeding the indicator line L can be accommodated by the expanded capacity resulting from the deformation of the elastic component 14. It can be seen that the combination of the indicator line L with the clastic component 14 in the present application can better improve the fault tolerance rate when the user adds absorbents, thereby improving efficiency for the user.

In some embodiments, when the height of the absorbent is higher than the indicator line L by a certain distance, and when the canister lid 12 is in the closed state, the absorbent presses the elastic component 14, thereby generating the external force F. For example, when the height of the absorbent is higher than the maximum indicator line L_max, which means that the absorbent exceeds the maximum capacity that can be accommodated in the accommodating chamber 111 of the absorber canister 100, and when the canister lid 12 is in the closed state, the absorbent presses the elastic component 14 to generate an action force, and said action force is the external force F.

In some embodiments, as shown in FIG. 1 to FIG. 3, the gas passage 13 includes a first gas hole 131, and the first gas hole 131 is disposed on the canister lid 12. For example, the first gas hole 131 may be a gas inlet hole, or a gas outlet hole.

In some embodiments, as shown in FIG. 1 and FIG. 2, the absorber canister 100 further includes a handle 15, the handle 15 includes an internal passage 151 and a second gas hole 152, and a lower end of the internal channel 151 is in communication with a lower end of the accommodating chamber 111. The first gas hole 131, the accommodating chamber 111, the internal passage 151, and the second gas hole 152 communicate in sequence to form the gas passage 13.

The inventors have realized that the structure of the absorber canister 100 is generally compact. It is challenging to provide a gas passage in the compact absorber canister 100 to ensure that the gas flow can sufficiently pass through the absorbent in the absorber canister 100. In the present application, by providing the internal passage 151 in the handle 15, there is no need to provide a dedicated gas passage to match the first gas hole on the canister lid 12, which saves space to a large extent. In addition, the handle 15 is relatively long, so that the internal passage 151 can have a certain buffering effect on the gas flow, thereby further helping bring the gas to be absorbed into full contact with the absorbent in the absorber canister 100, and improving the absorption effect. In addition, the second gas hole 152 of the handle 15 is disposed at the same end as the first gas hole 131, i.e., at an upper end of the absorber canister 100, so that liquid water generated in the absorption process does not flow out through the first gas hole 131 or the second gas hole 152 into the entire gas passage in which the absorber canister 100 is located.

In an embodiment of the present application, the internal passage 151 and the second gas hole 152 on the handle 15 may be configured in a variety of manners.

For example, the handle 15 may have a hollow structure, the second gas hole 152 is provided on an upper end of the handle 15, a lower end of the handle 15 is in communication with a lower end of the canister body 11, and the handle 15 may be integrally formed with the canister body 11.

For another example, the second gas hole 152 may be a gas inlet hole, or a gas outlet hole. One of the first gas hole 131 and the second gas hole 152 is a gas inlet hole, and the other is a gas outlet hole. If the first gas hole 131 is a gas inlet hole, then the second gas hole 152 is a gas outlet hole. If the first gas hole 131 is a gas outlet hole, then the second gas hole 152 is a gas inlet hole. Gas, such as carbon dioxide (CO2) exhaled by a patient, passes through the first gas hole 131, the accommodating chamber 111, the internal passage 151, and the second gas hole 152 in sequence, so as to enter the absorber canister 100, and then passes through the absorbent and flows out of the absorber canister 100.

It will be appreciated that, in addition to providing a passage for gas, the handle 15 also provides the user with the function of gripping the absorber canister 100. This configuration is more conducive to user operation without increasing the overall volume of the absorber canister 100.

In some embodiments, the canister lid 12 may be closed on the canister body 11 in a manner shown in FIG. 3. An end of the canister lid 12 is rotatably connected to the canister body 11 by means of a rotating shaft 22. A latch 23 is provided at an end opposite to the rotating shaft 22. In this way, after being snapped onto the canister body 11, the canister lid 12 can engage with the canister body 11 by means of the latch 23. It will be appreciated that any other closing manner is allowed, for example, the canister lid 12 and the canister body 11 may be engaged together by means of a plurality of latches, or may be engaged together by means of a threaded connection. The present application does not place limitations thereon.

As described above in the present application, in the embodiments of the present application, the elastic component 14 may be disposed on the canister body 11 or the canister lid 12, or both the canister body 11 and the canister lid 12 may be provided with the elastic component 14. The manner of configuring the elastic component is described in more detail below.

First, embodiments in which the elastic component 14 may be disposed on the canister body 11 are described.

In some embodiments, as shown in FIG. 2 and FIG. 3, a bottom mesh 16 is provided at the bottom of the accommodating chamber 111, and the bottom mesh 16 is configured to prevent the absorbent from passing through while allowing the gas flow to pass through.

For example, the bottom mesh 16 is disposed at the bottom of the canister body 11 or the accommodating chamber 111, a plurality of holes 161 are distributed on the bottom mesh 16, and the diameters of the plurality of holes 161 are smaller than the diameters of the particles of the absorbent, thereby allowing the gas flow to pass through and preventing the absorbent from passing through, that is, preventing the absorbent from leaking beneath the bottom mesh 16.

In some embodiments, the clastic component 14 includes a bottom elastic component 141 disposed between underneath the bottom mesh 16 and the bottom of the accommodating chamber 111, and the bottom elastic component 141 is compressed by the external force to lower the bottom mesh 16, so as to increase the volume of the accommodating chamber 111.

For example, the bottom elastic component 141 is disposed in a space between the bottom mesh 16 and the bottom of the accommodating chamber 111, and when the accommodating chamber 111 is gradually being filled with the absorbent, pressure borne by the bottom mesh 16 gradually increases, thereby pressing the bottom elastic component 141, that is, acting on the bottom elastic component with an external force. The bottom elastic component 141 is deformed and compressed by the external force, thereby lowering the bottom mesh 16. As described above in the present application, the elasticity of the bottom elastic component 141 is configured such that the weight of the absorbent is offset, but there is still room left for the bottom elastic component 141 to be compressed by the external force to continuously deform downward. In such a configuration, even if the weight of the absorbent causes deformation of the bottom elastic component 141, the bottom elastic component 141 can continue to be compressed when being pressed by the canister lid subsequently, thereby increasing the volume of the accommodating chamber 111 and preventing the canister lid from being unable to close.

It will be appreciated that the resilient force that can be provided by the bottom elastic component 141 is determined by a stiffness coefficient of an elastic material thereof (e.g., a spring). Through selection of an elastic material with a proper stiffness coefficient, it can be ensured that when deformation caused by compression has not yet reached the maximum value, the provided resilience force is already sufficient to support the filled absorbent, thereby preventing the bottom elastic component 141 from being compressed to its limit value solely under the weight of the absorbent.

In some embodiments, a rigid support component 17 is further disposed under the bottom mesh 16, the length of the rigid support component 17 is less than the length of the bottom elastic component 141, and the rigid support component 17 is not in contact with a bottom surface of the canister body 11 when the bottom elastic component 141 is not compressed. When the bottom elastic component 141 is compressed by a certain distance, the rigid support component 17 contacts the bottom surface of the canister body 11 to prevent the bottom elastic component 141 from being further compressed.

For example, the rigid support component 17 is integrally formed with the bottom mesh 16. The length of the rigid supporting member 17 is less than the natural length of the bottom elastic component 141. The natural length of the bottom elastic component 141 means the length of the bottom elastic component 141 when not being compressed, or the original length of the bottom elastic component 141 when no external force is applied thereto. The rigid support component 17 is provided to prevent the bottom elastic component 141 from being excessively compressed, and to form a space at the bottom of the canister body 11 to ensure smooth passage of gas and to store liquid water generated by chemical reaction.

In some embodiments, the bottom elastic component 141 includes one or more springs. Alternatively, the bottom elastic component 141 includes one or more plastic members or elastic members, and the plastic members or elastic members have a bent structure.

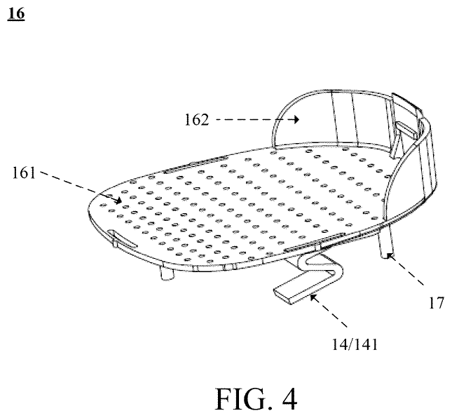

For example, the bottom elastic component 141 may be a spring or a plastic member. As shown in FIG. 3, the bottom elastic component 141 is a spring. As shown in FIG. 4, the bottom elastic component 141 is a plastic member. When the bottom elastic component 141 is a plastic member, the bottom clastic component may be integrally formed with the bottom mesh 16. There may be one or more bottom elastic components 141. When there is one bottom clastic component 141, the bottom elastic component may be disposed at a middle position under the bottom mesh 16. When there are a plurality of bottom elastic components 141, the plurality of bottom elastic components may be uniformly disposed under the bottom mesh 16.

In some embodiments, as shown in FIG. 2 and FIG. 3, one or more guide portions 18 are further disposed in the canister body 11, and the guide portions 18 guide the movement of the bottom mesh 16 in the vertical direction. For example, the guide portion 18 may be a guide pin.

In one example, the size of the accommodating chamber 111 includes a structure gradually enlarging from bottom to top in the vertical direction, and such an arrangement is more advantageous for removing and installing the bottom mesh, thereby facilitating replacement or cleaning and disinfection. In another example, the bottom mesh 16 and the bottom elastic component 141 may be connected together, for example, by integral formation or snap-fitting, making it easier to remove them together.

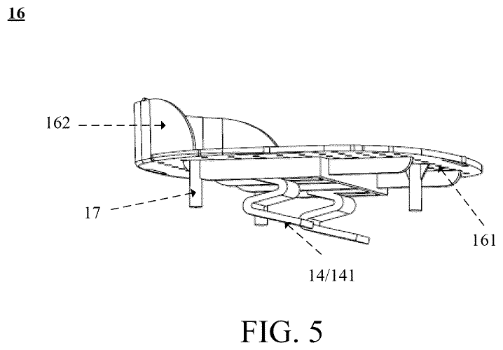

Then, referring to FIG. 4 and FIG. 5, FIG. 4 is a schematic diagram of the bottom mesh 16, and FIG. 5 is another schematic diagram of the bottom mesh 16.

In some embodiments, as shown in FIG. 4 and FIG. 5, the bottom mesh 16 further includes a wall 162 that fits an inner surface of the canister body 11, the shape of the wall 162 matches the shape of the inner surface of the canister body 11, and the wall 162 extends upwardly by a certain distance to block a gap between the internal passage 151 and the inner surface of the canister body 11 when the bottom mesh 16 is lowered down.

For example, since the canister body 11 is in communication with the handle 15, that is, the internal passage 151 is in communication with the accommodating chamber 111, there is a gap G between the canister body 11 and the handle 15, and the gap G is located on the inner surface of the canister body 11. The shape of the wall 162 matches the shape of the gap G. During the process of filling the canister body 11 or the accommodating chamber 111 with the absorbent, the bottom mesh 16 is gradually lowered, and when the bottom mesh 16 is lowered to the height of the gap G, the wall 162 can block the gap G, thereby avoiding leakage of the absorbent. In addition, the wall 162 further has a guiding function to ensure smooth vertical movement of the bottom mesh 16, making the bottom mesh less likely to turn over. This configuration can prevent the bottom elastic component 141 from being inclined and thus jammed during vertical movement, thereby further ensuring the reliability of the bottom elastic component 141 during use.

The embodiments in which the elastic component 14 may be disposed on the canister body 11 are described above. Embodiments in which the elastic component 14 may be disposed on the canister lid 12 are described below. It should be noted that, as described above in the present application, the manners of configuring the elastic component 14 may be freely combined in light of the teaching of the present application, for example, the clastic component 14 being disposed on only one of the canister lid 12 and the bottom of the canister body 11, or being disposed on both.

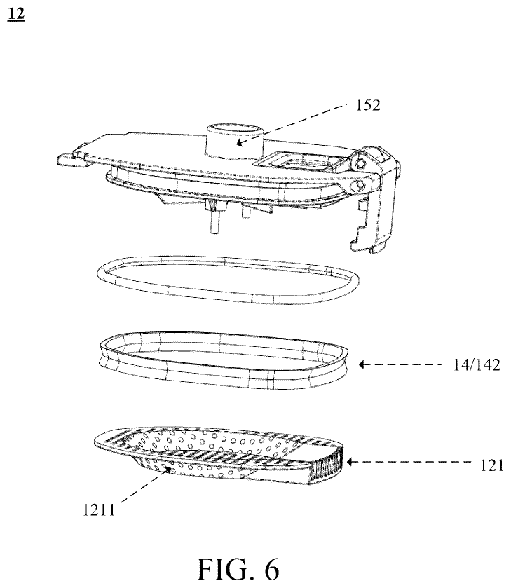

FIG. 6 is a schematic diagram of the canister lid 12.

In some embodiments, as shown in FIG. 6, the bottom of the canister lid 12 is provided with a top mesh 121, and the top mesh 121 is configured to allow the gas flow to pass through and prevent the absorbent from passing through.

For example, a plurality of holes 1211 are uniformly distributed on the top mesh 121, and the diameters of the plurality of holes 1211 are smaller than the diameters of the particles of the absorbent, thereby allowing the gas flow to pass through and preventing the absorbent from passing through, that is, preventing the absorbent from leaking onto the top mesh 121.

In some embodiments, as shown in FIG. 6, the elastic component 14 includes a top elastic component 142 disposed between the canister lid 12 and the top mesh 121, and the top elastic component 142 is compressed by the external force to raise the top mesh 121, so as to increase the volume of the accommodating chamber 111.

For example, the top elastic component 142 is disposed in a space between the canister lid 12 and the top mesh 121, and when the accommodating chamber 111 is filled with a large amount of absorbent, after the canister lid 12 is closed, the absorbent will press the top elastic component 142, that is, acting on the top elastic component with an external force, and the top elastic component 142 is deformed and compressed by the external force. As a result, the top mesh 121 is raised, thereby increasing the volume of the accommodating chamber 111.

In some embodiments, the canister lid 12 is further provided with one or more guide pins 122, and the guide pins 122 pass through the top mesh 121 to guide the movement of the top mesh 121 in the vertical direction.

In some embodiments, the top elastic component 142 includes one or more springs. Alternatively, the top elastic component 142 includes one or more elastic members (for example, elastic rubber). Alternatively, the top elastic component 142 includes one or more plastic members.

For example, the top elastic component 142 may be a spring, a plastic member, or elastic rubber. There may be one or more top elastic components 142. When there is one top elastic component 142, the top elastic component may be disposed at a middle position above the top mesh 121. When there are a plurality of top elastic components 142, the plurality of top elastic components may be uniformly disposed above the top mesh 121.

According to any of the above embodiments of the present application or a combination thereof, the elastic component is disposed in the absorber canister, so that the volume of the accommodating chamber for accommodating the absorbent can be changed. Even if a user fills the absorber canister with too much absorbent, the canister lid can still be closed properly. In this way, it is convenient for the user to fill the absorber canister with absorbent, and the user experience is improved. The absorber canister in the above embodiments is applicable to an arbitrary medical device (e.g., an anesthesia machine) in the art. For the specific configuration of the anesthesia machine, reference may be made to the structure of any anesthesia machine in the art. The present application does not place limitations thereon, and the following description is merely exemplary.

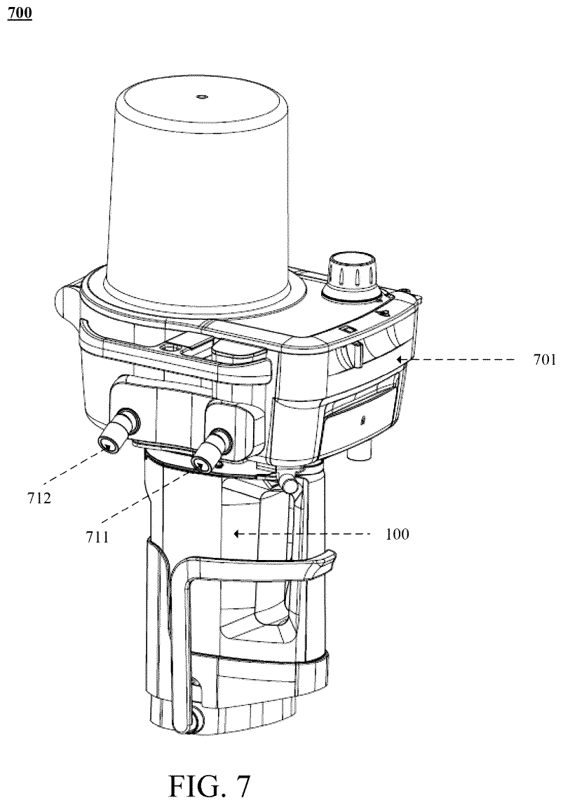

The embodiments of the present application further provide a medical device 700. FIG. 7 is a schematic diagram of the medical device 700. As shown in FIG. 7, the medical device 700 has a breathing circuit 701, and the absorber canister 100 described above is disposed on the breathing circuit 701.

In some examples, the breathing circuit 701 includes a first gas passage 711 and a second gas passage 712. After the absorber canister 100 is installed on the breathing circuit 701, the first gas passage and the second gas passage can be connected to the first gas hole and the second gas hole described in any of the above embodiments of the present application, respectively. In this way, the absorber canister 100 can be integrated into the breathing circuit 701 to form a complete gas passage circuit. Thus, the absorber canister 100 can perform the function of absorbing gas in the circuit. For example, the absorber canister 100 can connect to the airway of a patient via the breathing circuit 701 to absorb carbon dioxide and water vapor in the patient's lung gases and allow the remaining gas after absorption to continue flowing into the breathing circuit for the next cycle.

It will be appreciated that FIG. 7 of the present application illustrates only key components of the medical device 700 (e.g., an anesthesia machine). The other components of the anesthesia machine may be any components of an existing anesthesia machine in the art, and will not be described in detail here.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred implementations of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the implementations of the present application are not limited to the precise structures and operations illustrated and described, but can encompass all appropriate modifications, changes, and equivalents that fall within the scope of the implementations.

1. An absorber canister, comprising:
a canister body including an accommodating chamber for accommodating an absorbent, wherein an opening is provided at an upper end of the canister body;
a canister lid connected to the canister body, wherein the canister lid, in a closed state, closes the opening;
a gas passage including the accommodating chamber, wherein a gas flow can flow through the absorbent in the accommodating chamber via the gas passage; and
an elastic component disposed in the accommodating chamber, wherein the elastic component is deformable by an external force to increase the volume of the accommodating chamber.

2. The absorber canister according to claim 1, wherein the canister body is further provided with an indicator line, the indicator line being used to indicate the height of the absorbent.

3. The absorber canister according to claim 2, wherein when the height of the absorbent is higher than the indicator line by a certain distance, the canister lid presses the absorbent in the closed state, to generate the external force.

4. The absorber canister according to claim 1, wherein the gas passage includes a first gas hole, and the first gas hole is provided on the canister lid.

5. The absorber canister according to claim 4, wherein the absorber canister further includes a handle, the handle includes an internal passage and a second gas hole, and a lower end of the internal passage is in communication with a lower end of the accommodating chamber; and the first gas hole, the accommodating chamber, the internal passage, and the second gas hole communicate in sequence to form the gas passage.

6. The absorber canister according to claim 1, wherein a bottom mesh is provided at the bottom of the accommodating chamber, the bottom mesh being configured to allow the gas flow to pass through and prevent the absorbent from passing through.

7. The absorber canister according to claim 6, wherein the elastic component includes a bottom elastic component disposed between underneath the bottom mesh and the bottom of the accommodating chamber, and the bottom elastic component is compressed by the external force, so as to lower the bottom mesh and increase the volume of the accommodating chamber.

8. The absorber canister according to claim 7, wherein a rigid support component is further disposed under the bottom mesh, and the length of the rigid support component is less than the length of the bottom elastic component, and wherein when the bottom elastic component is not compressed, the rigid support component is not in contact with a bottom surface of the canister body; and when the bottom elastic component is compressed by a certain distance, the rigid support component contacts the bottom surface of the canister body to prevent the bottom elastic component from being further compressed.

9. The absorber canister according to claim 7, wherein the bottom elastic component includes one or more springs or the bottom elastic component includes one or more plastic members or elastic members, and the plastic members or elastic members have a bent structure.

10. The absorber canister according to claim 7, wherein one or more guide portions are further provided in the canister body, the guide portions guiding the movement of the bottom mesh in the vertical direction.

11. The absorber canister according to claim 6, wherein the bottom mesh includes a wall that fits an inner surface of the canister body, the shape of the wall matches the shape of the inner surface of the canister body, and the wall extends upward by a certain distance to block a gap between the internal passage and the inner surface of the canister body when the bottom mesh is lowered down.

12. The absorber canister according to claim 1, wherein a top mesh is provided at the bottom of the canister lid, the top mesh being configured to allow the gas flow to pass through and prevent the absorbent from passing through.

13. The absorber canister according to claim 12, wherein the elastic component includes a top elastic component disposed between the canister lid and the top mesh, and the top elastic component is compressed by the external force to raise the top mesh, so as to increase the volume of the accommodating chamber.

14. The absorber canister according to claim 13, wherein the canister lid is further provided with one or more guide pins, and the guide pins pass through the top mesh to guide the movement of the top mesh in the vertical direction.

15. The absorber canister according to claim 13, wherein the top elastic component includes one or more springs or the top elastic component includes one or more elastic members or plastic members.
